(19) United States
(12) Patent Application Publication
(10) Pub. No.: US 2023/0312874 A1
(43) Pub. Date: Oct. 5, 2023

(54) COMPOUND, RUBBER MIXTURE CONTAINING THE COMPOUND, VEHICLE TIRE WHICH HAS AT LEAST ONE COMPONENT COMPRISING THE RUBBER MIXTURE, METHOD FOR PRODUCING THE COMPOUND, AND USE OF THE COMPOUND AS AN AGING PROTECTION AGENT AND/OR ANTIOXIDANT AGENT AND/OR ANTIOZONANT AND/OR DYE

(71) Applicant: Continental Reifen Deutschland GmbH, Hannover (DE)

(72) Inventors: Andreas Jacob, Hannover (DE); David-Raphael Dauer, Dassel (DE); Julian Strohmeier, Einbeck (DE); Anna-Lena Dreier, Hannover (DE); Carla Recker, Hannover (DE); Jörg-August Becker, Hannover (DE); Florian Matz, Hannover (DE); Rebecca Graf, Hannover (DE); Jan Flormann, Hannover (DE)

(73) Assignee: Continental Reifen Deutschland GmbH, Hannover (DE)

(21) Appl. No.: 18/247,163

(22) PCT Filed: Sep. 29, 2021
(86) PCT No.: PCT/DE2021/200132

§ 371 Date: Mar. 29, 2023

(30) Foreign Application Priority Data
Oct. 2, 2020 (DE) 10 2020 212 507.4

(51) Int. Cl. C08K 5/18; C08L 21/00; C07C 213/06

(52) U.S. Cl. CPC C08K 5/18; C08L 21/00; C07C 213/06

(57) ABSTRACT

The invention relates to a compound, to a rubber mixture containing the compound, to a vehicle tire comprising the rubber mixture in at least one component, to a process for producing the compound and to the use of the compound as an aging stabilizer and/or antioxidant and/or dye.

The compound according to the invention has the formula I).

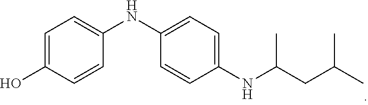 [SMILES: CC(C)CC(C)Nc1ccc(Nc2ccc(O)cc2)cc1]

The invention relates to a compound, to a rubber mixture containing the compound, to a vehicle tire comprising the rubber mixture in at least one component, to a process for producing the compound and to the use of the compound as an aging stabilizer and/or antioxidant and/or antiozonant and/or dye.

It is known that vehicle tires and technical rubber articles employ polymeric materials such as especially rubbers.

In case of prolonged storage and especially in the target application, which is often at elevated temperatures, natural rubber and synthetic polymers (such as IR, BR, SBR, ESBR, etc.), but also natural and synthetic oils, fats and lubricants, are subject to oxidation reactions which have an adverse effect on the original, desired properties. Depending on the type of the polymer, the polymer chains are shortened right up to the liquefaction of the material or subsequent hardening of the material occurs.

Aging stabilizers thus play a decisive role in the durability of vehicle tires and other technical rubber articles.

Known aging stabilizers are aromatic amines, for example 6-PPD (N -(1,3-dimethylbutyl)-N′-phenyl-p-phenylenediamine), IPPD (N-isopropyl-N′-phenyl-p -phenylenediamine) or SPPD (N-(1-phenylethyl)-N′-phenyl-p-phenylenediamine).

These molecules can react with oxygen or ozone or free radicals formed, such as alkyl, alkoxy and alkylperoxy radicals, and thus scavenge these and accordingly protect the rubbers etc. from further oxidation reactions.

Aging stabilizers which especially react with ozone and effect scavenging thereof are also referred to as “antiozonants”.

It is an object of the invention to provide a novel compound usable in particular as an aging stabilizer in vehicle tires or other technical rubber articles in order thus to achieve an improved protective effect on these articles based on the prior art.

The object is achieved by the inventive compound according to claim 1, by the inventive rubber mixture containing the compound and also by the inventive vehicle tire comprising the inventive rubber mixture in at least one component. The object is further achieved by the process for producing the compound and by the use of the compound as an aging stabilizer and/or antioxidant and/or antiozonant.

The compound according to claim 1 may further be used as a dye.

The compound according to claim 1 has the formula I):

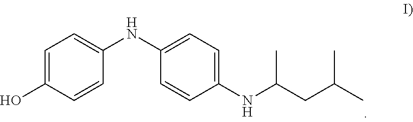 [SMILES: CC(C)CC(C)Nc1ccc(Nc2ccc(O)cc2)cc1]

The compound of formula I) is accordingly N-(1,3-dimethylbutyl)-N′-p -hydroxyphenyl-p-phenylenediamine.

Compared to the known aging stabilizers 6PPD or IPPD, the compound of formula I) presumably exhibits increased reactivity towards oxygen, ozone or free radicals (see above) due to the hydroxyl group, as a result of which the compound of formula I) achieves an improved protective effect, in particular in vehicle tires and other technical rubber articles, but also in oils and lubricants.

However, the invention shall not be be bound to a particular mechanism of action or a particular explanation.

DE1220124 discloses a compound of formula S1):

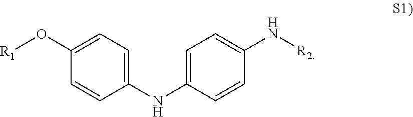

Compared to the compound of formula S1), the inventive compound of formula I) has a further hydrogen donor in the form of the hydroxyl group, thus allowing the compound of formula I) to scavenge more free radicals.

Combinations of different aging stabilizer classes, for example phenols with aromatic amines, are also known from CN 105272892 and CN 107935867. However, the individual units are not in direct conjugation, but rather are always isolated by an aliphatic spacer. There is thus no direct influencing/interaction between the groups. The inventive compound of formula I) therefore likewise has an improved reactivity and thus protective effect compared to the compounds from CN 105272892 and CN 107935867.

U.S. Pat. No. 6,770,785B1 discloses the compounds of formula S2 and S3.

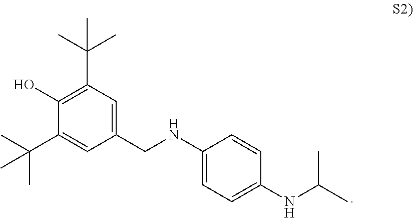 [SMILES: CC(C)Nc1ccc(NCc2cc(C(C)(C)C)c(O)c(C(C)(C)C)c2)cc1]

Compared to the compound of formula S2), the inventive compound of formula I) has the advantage that its units are conjugated, thus resulting in a better protective effect. In formula S2), the conjugation is interrupted by the —CH₂—NH moiety.

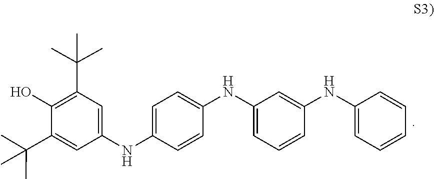 [SMILES: CC(C)(C)c1cc(Nc2ccc(Nc3cccc(Nc4ccccc4)c3)cc2)cc(C(C)(C)C)c1O]

Compared to the compound of formula S3), the inventive compound of formula I) has the advantage that the outer nitrogen atom bears an aliphatic and not an aromatic group, thus resulting in a better protective effect against ozone.

Compounds/molecules which have only aliphatic or aromatic radicals on the outer nitrogen atom instead of the 1,3-dimethylbutyl group and a hydrogen atom, i.e. no hydrogen atom, and are therefore maximally substituted on the nitrogen atom, likewise exhibit poorer protection against ozone compared to the inventive compound according to formula I) since this portion of the molecule has a decisive influence on reactivity and the possibility of elimination of a hydrogen atom from the nitrogen atom co-determines the protective effect.

The inventive compound of formula I) is particularly suitable as an aging stabilizer and/or antiozonant in vehicle tires and/or technical rubber articles, such as in particular an air spring, bellows, conveyor belt, belt, drive belt, hose, rubber band, profile, a seal, a membrane, tactile sensors for medical applications or robotics applications, or a shoe sole or parts thereof and/or oils and/or lubricants.

The inventive compound of formula I) is particularly suitable for producing a rubber article, in particular an air spring, a bellows, conveyor belt, belt, drive belt, hose, rubber band, profile, a seal, a membrane, tactile sensors for medical applications or robotics applications, or a shoe sole or parts thereof.

To use the compound according to formula I) in the recited articles or substances, said compound is used in a composition and used incorporated in said composition.

In vehicle tires or other technical rubber articles, this is in particular a rubber mixture.

The invention further provides for the use of the compound according to the invention of formula I) in oils and lubricants, such as in particular fuels or fluids for engines. The compound according to the invention may accordingly be employed in engines.

The present invention further provides for the use of the inventive compound according to formula I) as a dye in fibers and/or polymers and/or paper and/or in paints and coatings.

The present invention thus further provides a rubber mixture.

The rubber mixture according to the invention contains the compound of formula I). The rubber mixture according to the invention may in principle be any rubber mixture in which the novel inventive compound of formula I) achieves improved properties, in particular increased durability through aging stabilization and/or antiozonant effect.

The rubber mixture of the invention contains at least one rubber.

It is preferable when the rubber mixture according to the invention contains 0.1 to 10 phr, particularly preferably 0.1 to 5 phr, very particularly preferably 1 to 5 phr, of the compound of formula I).

The unit “phr” (parts per hundred parts of rubber by weight) used in this document is the conventional indication of quantity for mixture recipes in the rubber industry. The dosage of the parts by weight of the individual substances is based in this document on 100 parts by weight of the total mass of all high molecular weight (Mw greater than 20 000 g/mol) and hence solid rubbers present in the mixture.

In advantageous embodiments of the invention, the rubber mixture according to the invention contains at least one diene rubber.

The rubber mixture may accordingly contain a diene rubber or a mixture of two or more different diene rubbers.

Diene rubbers are rubbers which are formed by polymerization or copolymerization of dienes and/or cycloalkenes and thus have C═C double bonds either in the main chain or in the side groups.

The diene rubber is preferably selected from the group consisting of natural polyisoprene (NR), synthetic polyisoprene (IR), epoxidized polyisoprene (ENR), butadiene rubber (BR), butadiene-isoprene rubber, solution-polymerized styrene-butadiene rubber (SSBR), emulsion-polymerized styrene-butadiene rubber (ESBR), styrene-isoprene rubber, liquid rubbers having a molecular weight M_{w} of more than 20 000 g/mol, halobutyl rubber, polynorbornene, isoprene-isobutylene copolymer, ethylene-propylene-diene rubber, nitrile rubber, chloroprene rubber, acrylate rubber, fluororubber, silicone rubber, polysulfide rubber, epichlorohydrin rubber, styrene-isoprene-butadiene terpolymer, hydrogenated acrylonitrile butadiene rubber and hydrogenated styrene-butadiene rubber.

Nitrile rubber, hydrogenated acrylonitrile-butadiene rubber, chloroprene rubber, butyl rubber, halobutyl rubber and/or ethylene-propylene-diene rubber in particular are used in the production of technical rubber articles, such as belts, drive belts and hoses, and/or shoe soles. The mixture compositions known to those skilled in the art for these rubbers, which are specific in terms of fillers, plasticizers, vulcanization systems and additives, are preferably employed.

The natural and/or synthetic polyisoprene of all embodiments may be either cis-1,4-polyisoprene or 3,4-polyisoprene. However, the use of cis-1,4-polyisoprenes having a cis-1,4 proportion of >90% by weight is preferred. Such a polyisoprene is firstly obtainable by stereospecific polymerization in solution with Ziegler-Natta catalysts or using finely divided lithium alkyls. Secondly, natural rubber (NR) is one such cis-1,4-polyisoprene, for which the cis-1,4 content in the natural rubber is greater than 99% by weight.

A mixture of one or more natural polyisoprenes with one or more synthetic polyisoprenes is further also conceivable.

In the context of the present invention, the term “natural rubber” is to be understood as meaning naturally occurring rubber which may be obtained from Hevea rubber trees and from “non-Hevea” sources. Non-Hevea sources include for example Guayule shrubs and dandelion such as for example TKS (Taraxacum kok-saghyz; Russian dandelion).

If the rubber mixture of the invention contains butadiene rubber (i.e. BR, polybutadiene), this may be any of the types known to those skilled in the art. These include what are called the high-cis and low-cis types, with polybutadiene having a cis content of not less than 90% by weight being referred to as the high-cis type and polybutadiene having a cis content of less than 90% by weight being referred to as the low-cis type. An example of a low-cis polybutadiene is Li-BR (lithium-catalyzed butadiene rubber) having a cis content of 20% to 50% by weight. Particularly good properties and low hysteresis of the rubber mixture are achieved with a high-cis BR.

The polybutadiene(s) employed may be end group-modified with modifications and functionalizations and/or be functionalized along the polymer chains. The modification may be selected from modifications with hydroxyl groups and/or ethoxy groups and/or epoxy groups and/or siloxane groups and/or amino groups and/or aminosiloxane and/or carboxyl groups and/or phthalocyanine groups and/or silane-sulfide groups. However, other modifications known to those skilled in the art, also known as functionalizations, are also suitable. Metal atoms may be a constituent of such functionalizations.

In the case where at least one styrene-butadiene rubber (styrene-butadiene copolymer) is present in the rubber mixture, this may be either solution-polymerized styrene-butadiene rubber (SSBR) or emulsion-polymerized styrene-butadiene rubber (ESBR), a mixture of at least one SSBR and at least one ESBR also being employable. The terms “styrene-butadiene rubber” and “styrene-butadiene copolymer” are used synonymously in the context of the present invention.

The styrene-butadiene copolymer used may be end group-modified and/or functionalized along the polymer chains with the modifications and functionalizations recited above for the polybutadiene.

The at least one diene rubber is preferably selected from the group consisting of natural polyisoprene (NR, natural rubber), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

In a particularly preferred embodiment of the invention, the at least one diene rubber is selected from the group consisting of natural polyisoprene (NR), synthetic polyisoprene (IR), butadiene rubber (BR), solution-polymerized styrene-butadiene rubber (SSBR) and emulsion-polymerized styrene-butadiene rubber (ESBR).

In a particularly advantageous embodiment of the invention, the rubber mixture comprises at least one natural polyisoprene (NR), preferably in amounts of 5 to 55 phr, and in one particularly advantageous embodiment of the invention 5 to 25 phr, very particularly preferably 5 to 20 phr. Such a rubber mixture exhibits good processability and reversion stability and optimized tear properties and optimal rolling resistance characteristics.

In a particularly advantageous embodiment of the invention, the rubber mixture comprises at least one polybutadiene (BR, butadiene rubber), preferably in amounts of 10 to 80 phr, particularly preferably 10 to 50 phr, and in a particularly advantageous embodiment of the invention 15 to 40 phr. This achieves particularly good tear and abrasion properties of the rubber mixture according to the invention and optimal braking characteristics.

In a particularly advantageous embodiment of the invention, the rubber mixture comprises at least one solution-polymerized styrene-butadiene rubber (SSBR), preferably in amounts of 10 to 80 phr, particularly preferably 30 to 80 phr, and in one particularly advantageous embodiment of the invention 50 to 70 phr. This achieves particularly good rolling resistance properties of the rubber mixture according to the invention. In particularly advantageous embodiments of the invention, SSBR is employed in combination with at least one further rubber to achieve an optimal and balanced profile of properties.

It is preferable when the rubber mixture contains at least one filler, preferably in amounts of 30 to 500 phr, particularly preferably 50 to 400 phr, in turn preferably 80 to 300 phr.

In advantageous embodiments of the invention, the filler is a reinforcing filler which is preferably selected from the group consisting of carbon blacks and silicas.

In particularly advantageous embodiments of the invention, the rubber mixture contains at least one silica as filler, preferably in amounts of 30 to 500 phr, particularly preferably 50 to 400 phr, in turn preferably 80 to 300 phr.

In these quantities, silica is especially present as the sole or primary filler (more than 50% by weight based on total filler amount).

In further advantageous embodiments of the invention, the rubber mixture contains at least one silica as a further filler, preferably in amounts of 5 to 100 phr, particularly preferably 5 to 80 phr, in turn preferably 10 to 60 phr.

In these quantities, silica is especially present as a further filler in addition to another primary filler, such as in particular a carbon black.

The silica may be any of the types of silica known to those skilled in the art that are suitable as filler for tire rubber mixtures. However, particular preference is given to using a finely divided, precipitated silica which has a nitrogen surface area (BET surface area) (in accordance with DIN ISO 9277 and DIN 66132) of 35 to 400 m²/g, preferably 35 to 350 m²/g, more preferably 85 to 320 m²/g and most preferably 120 to 235 m²/g, and a CTAB surface area (in accordance with ASTM D 3765) of 30 to 400 m²/g, preferably 30 to 330 m²/g, more preferably 80 to 300 m²/g and most preferably 115 to 200 m²/g. Such silicas lead, for example in rubber mixtures for tire treads, to particularly good physical properties of the vulcanizates. Advantages in mixture processing by way of a reduction in mixing time can also result here while retaining the same product properties, leading to improved productivity. Silicas used may thus, for example, be either those of the Ultrasil® VN3 type (trade name) from Evonik or highly dispersible silicas known as HD silicas (e.g. Zeosil® 1165 MP from Solvay).

Where at least two different silicas, differing, for example, in their BET surface area, are present in the rubber mixture of the invention, the quantity figures stated refer to the total amount of all silicas present.

The terms “silicic acid” and “silica” are used synonymously in the context of the present invention.

The rubber mixture may additionally contain further fillers, such as in particular carbon blacks, in particular industrial carbon blacks or pyrolysis carbon blacks, or further reinforcing or non-reinforcing fillers.

Within the context of the present invention, the further (non-reinforcing) fillers include aluminosilicates, kaolin, chalk, starch, magnesium oxide, titanium dioxide, or rubber gels and also fibers (for example aramid fibers, glass fibers, carbon fibers, cellulose fibers).

Further, optionally reinforcing, fillers are for example carbon nanotubes (CNTs), including discrete CNTs, hollow carbon fibers (HCF) and modified CNTs containing one or more functional groups such as hydroxy, carboxy and carbonyl groups), graphite and graphenes and what is known as “carbon-silica dual-phase filler”.

In the context of the present invention, zinc oxide is not included among the fillers.

In particularly advantageous embodiments of the invention, the rubber mixture according to the invention contains 0.1 to 60 phr, preferably 3 to 40 phr, particularly preferably 5 to 30 phr, very particularly preferably 5 to 15 phr, of at least one carbon black. In these quantities, carbon black is present especially as a further filler in addition to a primary filler, such as in particular silica.

In further advantageous embodiments of the invention, the rubber mixture according to the invention contains 30 to 300 phr, preferably 30 to 200 phr, particularly preferably 40 to 100 phr, of at least one carbon black. In these quantities, carbon black is present as the sole or primary filler and is therefore optionally present in combination with silica in the abovementioned smaller amounts.

Suitable carbon blacks include any carbon black types familiar to those skilled in the art.

In one embodiment, the carbon black has an iodine number according to ASTM D 1510, also known as the iodine adsorption number, between 30 and 250 g/kg, preferably 30 to 180 g/kg, particularly preferably 40 to 180 g/kg, and very particularly preferably 80 to 150 g/kg, and a DBP number according to ASTM D 2414 of 30 to 200 ml/100 g, preferably 70 to 200 ml/100 g, particularly preferably 90 to 200 ml/100 g.

The DBP number in accordance with ASTM D 2414 determines the specific absorption volume of a carbon black or a light-colored filler by means of dibutyl phthalate.

The use of such a type of carbon black in the rubber mixture, in particular for vehicle tires, ensures the best possible compromise between abrasion resistance and heat buildup, which in turn influences the ecologically relevant rolling resistance. Preference is given here to only one type of carbon black being used in the respective rubber mixture, but it is also possible to mix various types of carbon black into the rubber mixture.

In a particularly advantageous embodiment of the invention, the rubber mixture contains 5 to 60 phr, particularly preferably 5 to 40 phr, of at least one carbon black and 50 to 300 phr, preferably 80 to 200 phr, of at least one silica.

The rubber mixture can further contain customary additives in customary parts by weight which are added preferably in at least one primary mixing stage during the production of said mixture. These additives include
a) aging stabilizers known in the prior art, for example diamines, such as N-Phenyl -N′-(1,3-dimethylbutyl)-p-phenylenediamine (6PPD), N, N′-diphenyl-p -phenylenediam ine (DPPD), N,N′-ditolyl-p-phenylenediamine (DTPD), N-(1,4-dimethylpentyl)-N′-phenyl-p-phenylenediamine (7PPD), N-isopropyl-N′-phenyl-p -phenylenediamine (IPPD), or dihydroquinolines, such as 2,2,4-trimethyl-1,2-dihydroquinoline (TMQ),
b) activators, for example zinc oxide and fatty acids (e.g. stearic acid) and/or other activators, such as zinc complexes, for example zinc ethylhexanoate,
c) activators and/or agents for binding fillers, in particular carbon black or silica, for example S-(3-aminopropyl)thiosulfuric acid and/or metal salts thereof (bonding of carbon black) and silane coupling agents (bonding of silica),
d) antiozonant waxes,
e) resins, especially tackifying resins for internal tire components,
f) masticating aids, for example 2,2′-dibenzamidodiphenyl disulfide (DBD), and
g) processing aids, such as in particular fatty acid esters and metal soaps, for example zinc soaps and/or calcium soaps,
h) plasticizers, such as in particular aromatic, naphthenic or paraffinic mineral oil plasticizers, for example MES (mild extraction solvate) or RAE (residual aromatic extract) or TDAE (treated distillate aromatic extract), or rubber-to-liquid oils (RTL) or biomass-to-liquid oils (BTL) preferably having a content of polycyclic aromatics of less than 3% by weight according to method IP 346 or triglycerides, for example rapeseed oil or factices or hydrocarbon resins or liquid polymers having a mean molecular weight (determination by GPC=gel permeation chromatography, in accordance with BS ISO 11344:2004) between 500 and 20 000 g/mol.

When using mineral oil, this is preferably selected from the group consisting of DAE (distillate aromatic extracts) and/or RAE (residual aromatic extract) and/or TDAE (treated distillate aromatic extracts) and/or MES (mild extracted solvents) and/or naphthenic oils.

In particularly advantageous embodiments, the rubber mixture according to the invention contains no further aging stabilizers from the group of p-phenylenediamines, see above list a), in addition to the inventive compounds of formula I). In a particularly preferred embodiment, the rubber mixture according to the invention especially contains 0 to 0.1 phr, in particular 0 phr, of further aging stabilizers based on diamines selected from the group containing, particularly preferably consisting of, N-phenyl-N′-(1,3-dimethylbutyl)-p-phenylenediamine (6PPD), N, N′-diphenyl-p-phenylenediam ine (DPPD), N, N′-ditolyl-p -phenylenediamine (DTPD), N-isopropyl-N′-phenyl-p-phenylenediamine (IPPD), N-(1,4-dimethylpentyl)-N′-phenyl-p-phenylenediamine (7PPD).

The preferably very small amounts of 0 to 0 phr, particularly preferably 0 phr, of the abovementioned diamines and of the compound of formula I) present according to the invention make it possible to achieve an improved protective effect. The inventive compound of formula I) replaces the recited phenylenediamines known in the prior art.

In further advantageous embodiments of the invention, at least one further representative of the recited diamine aging stabilizers is present and therefore the compound according to the invention only partially replaces the diamines known in the prior art. This also achieves the advantage according to the invention, just not to an optimal extent.

In advantageous embodiments, aging stabilizers based on dihydroquinoline, such as TMQ, are present in the rubber mixture in addition to the inventive compound of formula I). The amount of dihydroquinolines present, such as especially TMQ, is preferably 0.1 to 3, in particular 0.5 to 1.5 phr.

In further advantageous embodiments, the rubber mixture according to the invention contains no further aging stabilizers, i.e. 0 phr of further aging stabilizers other than the inventive compound of formula I).

The silane coupling agents may be any of the types known to those skilled in the art.

Furthermore, one or more different silane coupling agents may be used in combination with one another. The rubber mixture may thus contain a mixture of different silanes.

The silane coupling agents react with the surface silanol groups of the silica or other polar groups during the mixing of the rubber/the rubber mixture (in situ) or in the context of a pretreatment (premodification) even before addition of the filler to the rubber.

Coupling agents known from the prior art are bifunctional organosilanes having at least one alkoxy, cycloalkoxy or phenoxy group as a leaving group on the silicon atom and having as another functionality a group which, possibly after cleavage, can enter into a chemical reaction with the double bonds of the polymer. The latter group may for example comprise the following chemical groups:
—SCN, —SH, —NH₂ or —Sₓ— (with x=2 to 8).

Employable silane coupling agents thus include for example 3-mercaptopropyltriethoxysilane, 3-thiocyanatopropyltrimethoxysilane or 3,3′-bis(triethoxysilylpropyl) polysulfides having 2 to 8 sulfur atoms, for example 3,3′-bis(triethoxysilylpropyl) tetrasulfide (TESPT), the corresponding disulfide (TESPD), or else mixtures of the sulfides having 1 to 8 sulfur atoms with different contents of the various sulfides. TESPT may for example also be added as a mixture with carbon black (trade name X50S® from Evonik).

Blocked mercaptosilanes as known for example from WO 99/09036 may also be used as a silane coupling agent. It is also possible to use silanes as described in WO 2008/083241 A1, WO 2008/083242 A1, WO 2008/083243 A1 and WO 2008/083244 A1. It is also possible to use, for example, silanes which are marketed under the name NXT in a number of variants from Momentive, USA, or those which are marketed under the name VP Si 363® by Evonik Industries.

The total proportion of further additives is preferably 3 to 150 phr, more preferably 3 to 100 phr and most preferably 5 to 80 phr.

Zinc oxide (ZnO) may be included in the total proportion of the further additives in the abovementioned amounts.

This may be any type of zinc oxide known to those skilled in the art, for example ZnO granules or powder. The zinc oxide conventionally used generally has a BET surface area of less than 10 m²/g. However, it is also possible to use a zinc oxide having a BET surface area of 10 to 100 m²/g, for example so-called “nano zinc oxides”.

The rubber mixture according to the invention is preferably used in vulcanized form, in particular in vehicle tires or other vulcanized technical rubber articles.

The vulcanization of the rubber mixture of the invention is preferably conducted in the presence of sulfur and/or sulfur donors with the aid of vulcanization accelerators, it being possible for some vulcanization accelerators to act simultaneously as sulfur donors. The accelerator is selected from the group consisting of thiazole accelerators and/or mercapto accelerators and/or sulfenamide accelerators and/or thiocarbamate accelerators and/or thiuram accelerators and/or thiophosphate accelerators and/or thiourea accelerators and/or xanthogenate accelerators and/or guanidine accelerators.

Preference is given to using a sulfenamide accelerator selected from the group consisting of N-cyclohexyl-2-benzothiazolesulfenamide (CBS) and/or N,N-dicyclohexylbenzothiazole-2-sulfenamide (DCBS) and/or benzothiazyl-2-sulfenomorpholide (MBS) and/or N-tert-butyl-2-benzothiazylsulfenamide (TBBS) or a guanidine accelerator such as diphenylguanidine (DPG).

The sulfur donor substances used may be any sulfur donor substances known to those skilled in the art. If the rubber mixture contains a sulfur donor substance, the latter is preferably selected from the group comprising for example thiuram disulfides, for example tetrabenzylthiuram disulfide (TBzTD) and/or tetramethylthiuram disulfide (TMTD) and/or tetraethylthiuram disulfide (TETD), and/or thiuram tetrasulfides, for example dipentamethylenethiuram tetrasulfide (DPTT), and/or dithiophosphates, for example
DipDis (bis(diisopropyl)thiophosphoryl disulfide) and/or bis(O,O-2-ethylhexylthiophosphoryl) polysulfide (e.g. Rhenocure SDT 50®, Rheinchemie GmbH) and/or zinc dichloryldithiophosphate (e.g. Rhenocure ZDT/S®, Rheinchemie GmbH) and/or zinc alkyldithiophosphate, and/or 1,6-bis(N,N -dibenzylthiocarbamoyldithio)hexane and/or diaryl polysulfides and/or dialkyl polysulfides.

Further network-forming systems, such as can be obtained for example under the trade names
Vulkuren®, Duralink® or Perkalink®, or network-forming systems as described in WO 2010/049216 A2, can also be used in the rubber mixture. This system contains a vulcanizing agent which crosslinks with a functionality of greater than four and at least one vulcanization accelerator.

It is particularly preferable to use the accelerators TBBS and/or CBS and/or diphenylguanidine (DPG).

Vulcanization retarders may also be present in the rubber mixture.

The terms “vulcanized” and “crosslinked” are used synonymously in the context of the present invention.

In a preferred development of the invention, a plurality of accelerators are added in the final mixing stage during the production of the sulfur-crosslinkable rubber mixture.

The sulfur-crosslinkable rubber mixture of the invention is produced by the process that is customary in the rubber industry, in which, in one or more mixing stages, a base mixture comprising all constituents except for the vulcanization system (sulfur and vulcanization-influencing substances) is first produced. The finished mixture is produced by adding the vulcanization system in a final mixing stage. The finished mixture is for example processed further and brought into the appropriate shape by means of an extrusion operation or calendering.

This is followed by further processing by vulcanization, wherein sulfur crosslinking takes place due to the vulcanization system added within the context of the present invention.

The above-described rubber mixture of the invention is particularly suitable for use in vehicle tires, especially pneumatic vehicle tires.

For use in vehicle tires, the mixture, as a finished mixture prior to vulcanization, is preferably brought into the shape of a tread and is applied in the known manner during production of the green vehicle tire.

The production of the rubber mixture of the invention, for use as a sidewall or other body mixture in vehicle tires, is effected as has already been described. The difference lies in the shaping after the extrusion operation/the calendering of the mixture. The shapes thus obtained of the as-yet unvulcanized rubber mixture for one or more different body mixtures then serve for the construction of a green tire.

“Body mixture” refers here to the rubber mixtures for the inner components of a tire, such as essentially squeegee, inner liner (inner layer), core profile, belt, shoulder, belt profile, carcass, bead reinforcement, bead profile, flange profile and bandage. The as-yet unvulcanized green tire is subsequently vulcanized.

For use of the rubber mixture of the invention in drive belts and other belts, especially in conveyor belts, the extruded, as-yet unvulcanized mixture is brought into the appropriate shape and often provided at the same time or subsequently with strength members, for example synthetic fibers or steel cords. This usually affords a multi-ply construction consisting of one and/or more plies of rubber mixture, one and/or more plies of identical and/or different strength members and one and/or more further plies of the same and/or another rubber mixture.

The present invention further provides a vehicle tire comprising the rubber mixture according to the invention containing the compound according to the invention in at least one component.

The vulcanized vehicle tire comprises at least in one component a vulcanizate of at least one rubber mixture according to the invention. A person skilled in the art is aware that most of the substances, for example the rubbers present, are present or may be present in a chemically altered form either already after mixing or only after vulcanization.

Within the context of the present invention, “vehicle tires” are to be understood to mean pneumatic vehicle tires and solid rubber tires, including tires for industrial and construction site vehicles, truck, car and two-wheeled-vehicle tires.

The vehicle tire according to the invention preferably comprises the rubber mixture according to the invention in at least one external component, wherein the external component is preferably a tread, a sidewall and/or a flange profile.

The vehicle tire according to the invention may thus also comprise the rubber mixture according to the invention containing the inventive compound of formula I) in two or more components of optionally adapted composition.

The present invention further provides a process for producing the compound of formula I), wherein the process comprises at least the following process steps:
a) providing the substance of formula A)

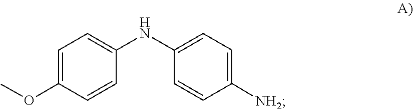 [SMILES: COc1ccc(Nc2ccc(N)cc2)cc1]

b) providing methyl isobutyl ketone (MIBK) and hydrogen or methyl isobutyl ketone (MIBK) and formic acid;
c) reacting the substance according to step a) with the substances according to step b), preferably in the presence of a hydrogenation catalyst, to afford the substance of formula C)

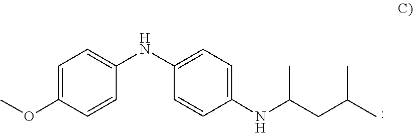 [SMILES: COc1ccc(Nc2ccc(NC(C)CC(C)C)cc2)cc1]

d) providing a Lewis acid, such as in particular boron tribromide (BBr₃), aluminum trichloride (AlCl₃), aluminum tribromide (AlBr₃), boron trifluoride diethyl etherate (BF₃*OEt₂) or tin tetrachloride (SnCl₄), or a Brønsted acid, such as in particular hydrogen iodide (HI) or hydrogen bromide (HBr);
e) reacting the substance of formula C) with the substance from step d) to afford the substance of formula I)

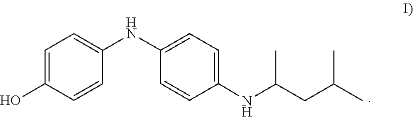 [SMILES: CC(C)CC(C)Nc1ccc(Nc2ccc(O)cc2)cc1]

In step b), the alternative methyl isobutyl ketone (MIBK) and hydrogen is preferred over MIBK and formic acid.

It is preferable when the substance of formula C) present in step c) is dissolved in an organic solvent, preferably an aprotic solvent, such as especially dichloromethane (DCM) or chloroform (CHCl₃), and then reacted according to step e).

The present invention further provides a further process for producing the compound of formula I), wherein the process comprises at least the following process steps:
a1) providing the substance of formula A)

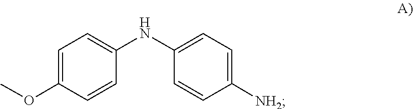 [SMILES: COc1ccc(Nc2ccc(N)cc2)cc1]

b1) providing a Lewis acid, such as in particular boron tribromide (BBr₃), aluminum trichloride (AlCl₃), aluminum tribromide (AlBr₃), boron trifluoride diethyl etherate (BF₃*OEt₂) or tin tetrachloride (SnCl₄), or a Brønsted acid, such as in particular hydrogen iodide (HI) or hydrogen bromide (HBr);
c1) reacting the substance according to step a1) with the substance from step b1) to afford the substance of formula C1)

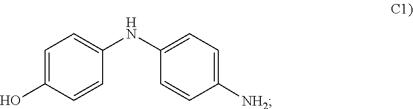 [SMILES: Nc1ccc(Nc2ccc(O)cc2)cc1]

d1) providing methyl isobutyl ketone (MIBK) and hydrogen;
e1) reacting the substance of formula C1) with the substances from step d1), preferably in the presence of a hydrogenation catalyst, to afford the substance of formula I)

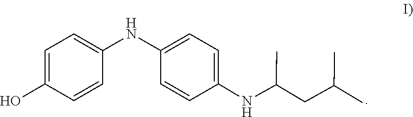 [SMILES: CC(C)CC(C)Nc1ccc(Nc2ccc(O)cc2)cc1]

It is preferable when the process steps in which a reaction with hydrogen is carried out employ a suitable catalyst, referred to in the context of the present invention as “hydrogenation catalyst”.

The hydrogenation catalyst of the processes (step c)/e1)) is preferably a noble metal catalyst, such as in particular palladium (Pd) or platinum (Pt). The noble metal is preferably employed on carbon (C), such as palladium on carbon (Pd/C).

It is also possible to employ other known catalysts, such as Raney nickel or copper chromite.

It is preferable when palladium on carbon (Pd/C) is used as hydrogenation catalyst in step c).

It is preferable when palladium on carbon (Pd/C) is used as hydrogenation catalyst in step e1).

The hydrogen pressure in the respective process steps in which hydrogen is used is preferably 1 to 40 bar, particularly preferably 10 to 30 bar, very particularly preferably 15 to 25 bar.

The hydrogenation reaction in step c)/e1) is preferably carried out in an autoclave, in particular a stainless steel autoclave.

The temperature in process steps c) and e1) is preferably from room temperature (RT, in particular 20° C.) to 100° C., particularly preferably 40° C. to 80° C., very particularly preferably 40° C. to 70° C.

Excessively high temperatures result in undesired hydrogenation of the aromatic constituents.

All process steps, in particular steps e) and c1), are advantageously carried out in a protective gas atmosphere.

Process steps e) and c1) are preferably carried out at a temperature of −78 (minus seventy-eight)° C. to +20° C., particularly preferably −10° C. to +10° C., in particular at 0° C.

The reactions in process steps e)/c1) are in each case an ether cleavage. In principle any substance with which the desired ether cleavage of substance C)/A) may be performed is suitable. The substance with which the ether cleavage is performed is in the context of the present invention also referred to as “ether cleavage reagent”.

Employable to this end is a Lewis acid, such as in particular boron tribromide (BBr₃), aluminum trichloride (AlC₁₃), aluminum tribromide (AlBr₃), boron trifluoride diethyl etherate (BF₃*OEt₂) or tin tetrachloride (SnCl₄), or a Brønsted acid such as in particular hydrogen iodide (HI) or hydrogen bromide (HBr),
Brønsted acids are familiar to those skilled in the art and may be characterized by means of their pKa value.

Strong Brønsted acids have a pKa of −1.74 (minus one point seven four) to 4.5 (four point five) and very strong acids have a pKa of less than −1.74, as also disclosed in Römpp Online Lexikon (URL: https://roempp.thieme.de/lexicon/RD-19-00110?searchterm=s%C3%A4ure).

In the context of the present invention, very strong Brønsted acids having a pKa of less than −1.74 are preferred, such as in particular hydrogen iodide (HI) or hydrogen bromide (HBr).

In particularly advantageous embodiments of the invention, c1) and e) employ a Lewis acid, in particular boron tribromide (BBr₃).

The substance of formula A) and boron tribromide (BBr₃) and methyl isobutyl ketone (MIBK) are commercially available. The other recited alternatives to BBr₃ are also commercially available.

The invention shall be more particularly elucidated below with reference to working examples.

The component of formula I) was produced as follows:

### X1): Synthesis of N-(1,3-dimethylbutyl)-N′-p-methoxyphenyl-p-phenylenediamine (compound of formula C)) according to step c)

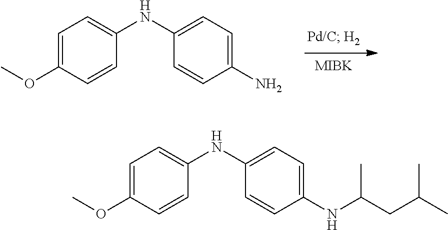 [SMILES: COc1ccc(Nc2ccc(N)cc2)cc1 ; COc1ccc(Nc2ccc(NC(C)CC(C)C)cc2)cc1]

5.00 g (23.3 mmol, 1 eq.) of 4-(4-methoxyphenylamino)aniline, 2.00 g of Pd/C (palladium on carbon C) (5%) (0.2 g on 4.67 mmol of substrate) and 50.0 mL of methyl isobutyl ketone (MIBK) were weighed into a stainless steel autoclave fitted with a Teflon inliner. The autoclave was then pressurized to 20 bar with hydrogen (H₂) and stirred at 60° C. for 10 hours. After termination of the reaction, the excess hydrogen was blown off and the suspension filtered through diatomaceous earth (Celite®) and washed with ethanol. The filtrate was concentrated to dryness and dried under vacuum. The residue was purified over silica gel (cyclohexane/acetic ester 100:0→80:20). Orange oil; yield 5.6 g (80% of theory).

¹H-NMR (500 MHz, DMSO-d6) δ=8.09 (s, 1H), 6.94 (td, J=7.6, 1.5 Hz, 1 H), 6.88 (dd, J=7.6, 1.4 Hz, 1 H), 6.69-6.63 (m, 2H), 6.51 (d, J=8.4 Hz, 1 H), 6.27 (dd, J=8.5, 2.5 Hz, 1H), 6.21 (d, J=2.5 Hz, 1H), 4.82 (d, J=8.9 Hz, 1H), 3.30 (dt, J=8.6, 6.5 Hz, 1H), 1.70 (dp, J=13.5, 6.7 Hz, 1H), 1.38 (dt, J=13.9, 7.1 Hz, 1H), 1.16 (dt, J=13.5, 6.8 Hz, 1H), 1.02 (d, J=6.1 Hz, 3H), 0.87 (dd, J=19.4, 6.6 Hz, 6H).

¹³C-NMR (126 MHz, DMSO-d6) δ=151.8, 143.0, 140.0, 133.0, 120.7, 116.0, 114.5, 113.6, 55.2, 46.1, 45.9, 26.3, 24.5, 22.8, 22.6, 20.87 (46.1 and 45.9 is splitting due to enantiomers).

ESI-MS (electrospray ionization mass spectrometry) [M+H]⁺=299.

### Synthesis of N-(1,3-dimethylbutyl)-N′-p-hydroxyphenyl-p-phenylenediamine (inventive compound of formula I)) according to step e)

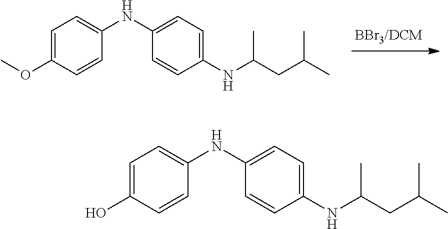 [SMILES: COc1ccc(Nc2ccc(NC(C)CC(C)C)cc2)cc1 ; CC(C)CC(C)Nc1ccc(Nc2ccc(O)cc2)cc1]

Under argon, 5.70 g (19.1 mmol, 1 eq.) of N-(1,3-dimethylbutyl)-N′-p -methoxyphenyl-p-phenylenediamine were dissolved in 20.0 mL of dry dichloromethane (DCM) and cooled to 0° C. 5.52 mL (57.3 mmol, 5 eq.) of boron tribromide were dissolved in 15 mL of dry DCM and carefully added dropwise. The mixture was stirred overnight and the reaction was terminated under protective gas and with ice cooling by addition of saturated NaHCO₃ solution. 50 mL of a 3:1 mixture of acetic acid/isopropanol were additionally added. The organic phase was washed with saturated NaHCO₃ solution and saturated NaCl solution and dried over MgSO₄. Once filtration was complete, the solvent was concentrated to dryness and dried under vacuum. Pale-violet to blue solid; yield 4.8 g (88% of theory).

¹H-NMR (500 MHz, DMSO-d6) δ=8.63 (s, 1H), 6.95 (s, 1H), 6.76 (d, J=8.7 Hz, 2H), 6.71 (d, J=8.8 Hz, 2H), 6.59 (d, J=8.7 Hz, 2H), 6.51-6.44 (m, 2H), 4.71 (d, J=8.2 Hz, 1 H), 3.40-3.31 (d, J=6.9 Hz, 1H), 1.73 (dp, J=13.4, 6.7 Hz, 1 H), 1.49-1.37 (m, 1H), 1.23-1.15 (m, 1H), 1.05 (d, J=6.1 Hz, 3H), 0.91 (d, J=6.6 Hz, 3H), 0.87 (d, J=6.6 Hz, 3H).

¹³C-NMR (126 MHz, DMSO-d6) δ=149.8, 142.5, 138.1, 133.9, 119.9, 117.0, 115.6, 113.3, 46.1, 45.9, 26.3, 24.5, 22.8, 22.6, 20.8 (46.1 and 45.9 is splitting due to isomers).

ESI-MS [M+H]⁺=285.

In an alternative variant of the reaction according to step c), the intermediate compound of formula C) was produced as follows:

### X2): Synthesis of N-(1,3-dimethylbutyl)-N′-p-methoxyphenyl-p-phenylenediamine

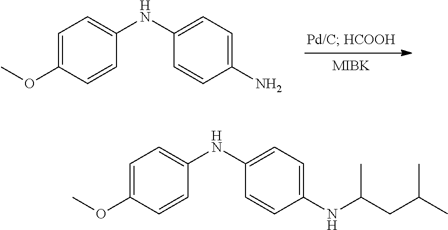 [SMILES: COc1ccc(Nc2ccc(N)cc2)cc1 ; COc1ccc(Nc2ccc(NC(C)CC(C)C)cc2)cc1]

Under protective gas, 0.40 g of palladium on carbon (5%) was suspended in 20 mL of dry ethanol and 1.00 g (4.67 mmol, 1 eq.) of 4-(4-methoxyphenylamino)aniline, 0.88 mL (23.3 mmol, 5 eq.) of formic acid (23.3 mmol, 5 eq.) and 1.17 mL (9.33 mmol, 2 eq.) of methyl isobutyl ketone were added successively. The mixture was heated for 4 hours under reflux, the solids were filtered off and the solvent was removed under vacuum. The residue was purified over silica gel (cyclohexane/acetic ester 100:0→80:20). Orange oil; yield 1.0 g (73% of theory).

The spectra are identical to the first performance (X1).

The performance of process step c) according to X1) is preferred due to the better reproducibility and fewer generated byproducts compared to that according to X2) (formate as byproduct from the reaction of amine and formic acid).

The inventive compound was also produced by an alternative synthesis route as follows:

### Synthesis of N-p-hydroxyphenyl-p-phenylenediamine (compound of formula C1) according to step c1)

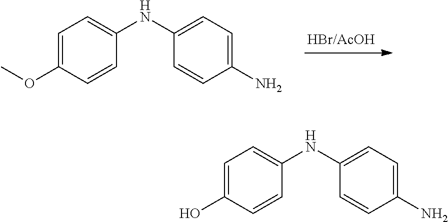 [SMILES: COc1ccc(Nc2ccc(N)cc2)cc1 ; Nc1ccc(Nc2ccc(O)cc2)cc1]

Under protective gas, 20 mL of degassed acetic acid and 10 mL of hydrobromic acid were initially charged and 1 g (4.67 mmol, 1 eq.) of 4-(4-methoxyphenylamino)aniline was dissolved therein and stirred at 120° C. for 24 hours. Once the solution had been brought to RT, it was neutralized with saturated NaHCO₃ solution and extracted with a 3:1 acetic ester/isopropanol mixture. The solvent was removed and the residue purified over silica gel (cyclohexane/acetic ester 1:1). Brown solid; yield 0.7 g (75% of theory).

¹H-NMR (500 MHz, DMSO-d6) δ=8.64 (s, 1H), 6.94 (s, 1H), 6.73-6.68 (m, 4H), 6.59 (d, J=8.8 Hz, 2H), 6.48 (d, J=8.6 Hz, 2H), 4.56 (s, 2H).

### Synthesis of (1,3-dimethylbutyl)-N′-p-hydroxyphenyl-p-phenylenediamine (inventive compound of formula I)) according to step e1)

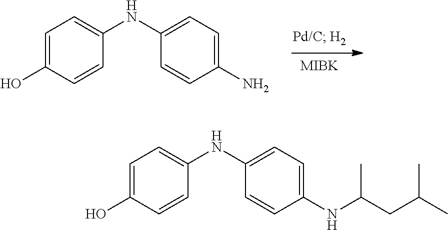 [SMILES: Nc1ccc(Nc2ccc(O)cc2)cc1 ; CC(C)CC(C)Nc1ccc(Nc2ccc(O)cc2)cc1]

0.31 g (1.55 mmol, 1 eq.) of N-p-hydroxyphenyl-p-phenylenediamine, 0.66 g of palladium on carbon (5%) (0.2 g on 4.67 mmol of substrate) and 20.0 mL of methyl isobutyl ketone were weighed into a stainless steel autoclave fitted with a Teflon innerliner. The autoclave was then pressurized to 20 bar with hydrogen and stirred at 60° C. for 10 hours. After termination of the reaction, the excess hydrogen was blown off and the suspension filtered through diatomaceous earth (Celite®) and washed with ethanol. The filtrate was concentrated to dryness and dried under vacuum. According to NMR analysis, the crude product contains 70% of the target molecule.

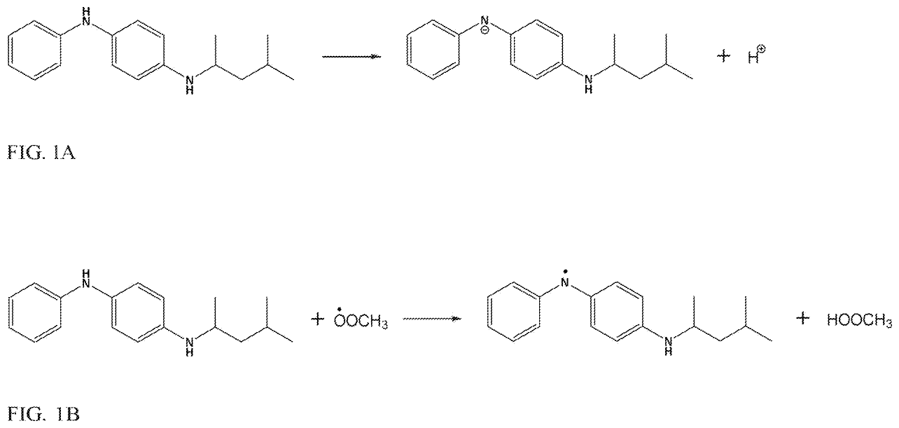

The inventive compound of formula I) exhibits an elevated reactivity relative to 6PPD. This was consistent for example with the calculation of binding dissociation energies (BDE), and the free activation enthalpy Δ_{R}G^{≠} and the free standard reaction enthalpy Δ_{R}G^{○} for the reaction with a methyl peroxide radical. The values are reported in table 1. FIGS. 1a and 1b show the cleavage mechanisms to which the values relate:

TABLE 1

| | BDE | Δ_{R}G^{O} | Δ_{R}G^{≠} |
|---|---|---|---|
| Molecule | [kJ/mol] | [kJ/mol] | [kJ/mol] |
| 6PPD | 313 | −25.6 | 19.1 |
| Formula I) | 305 | −28.8 | 16.0 |

As is apparent from table 1, the inventive compound of formula I) exhibits a lower bond dissociation energy and lower free enthalpies.

The compound of formula I) thus makes it possible to achieve an improved protective effect in the recited applications.

For use in a rubber mixture for vehicle tires, the inventive compound of formula I) is added for example instead of the aging stabilizers known in the prior art, such as 6PPD, 7PPD or IPPD etc., in a manner known to those skilled in the art in one of the mixing stages in the production of the rubber mixture.

1-12. (canceled)

13. A rubber mixture comprising a compound of formula I):

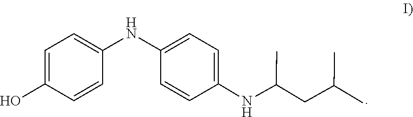 [SMILES: CC(C)CC(C)Nc1ccc(Nc2ccc(O)cc2)cc1]

14. The rubber mixture of claim 13, the mixture contains at least one diene rubber.

15. The rubber mixture of 3, wherein it contains at least one diene rubber selected from the group consisting of natural polyisoprene (NR), synthetic polyisoprene (IR), butadiene rubber (BR), solution-polymerized styrene-butadiene rubber (SSBR), emulsion-polymerized styrene-butadiene rubber (ESBR), butyl rubber (IIR), and halobutyl rubber.

16. The rubber mixture of claim 14, wherein the mixture is incorporated into a vehicle tire.

17. The rubber mixture of claim 16, the mixture is comprised in at least one external component of the tire, wherein the external component is a tread, a sidewall and/or a flange profile.

18. A method for producing a compound comprising:
a) providing the substance of formula A) 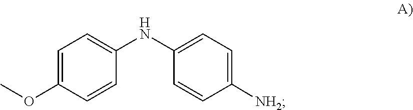 [SMILES: COc1ccc(Nc2ccc(N)cc2)cc1]
b) providing methyl isobutyl ketone (MIBK) and hydrogen or methyl isobutyl ketone (MIBK) and formic acid;
c) reacting the substance according to step a) with the substances according to step b) to afford the substance of formula C) 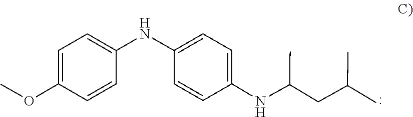 [SMILES: COc1ccc(Nc2ccc(NC(C)CC(C)C)cc2)cc1]
d) providing a Lewis acid, such as in particular boron tribromide (BBr₃), aluminum trichloride (AlCl₃), aluminum tribromide (AlBr₃), boron trifluoride diethyl etherate (BF₃*OEt₂) or tin tetrachloride (SnCl₄), or a Brønsted acid, such as in particular hydrogen iodide (HI) or hydrogen bromide (HBr);
e) reacting the substance of formula C) with the substance from step d) to afford the substance of formula I)

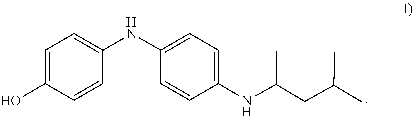 [SMILES: CC(C)CC(C)Nc1ccc(Nc2ccc(O)cc2)cc1]

19. A method for producing a compound comprising:
a1) providing the substance of formula A) 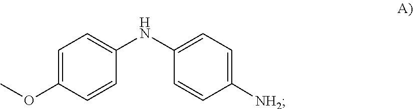 [SMILES: COc1ccc(Nc2ccc(N)cc2)cc1]
b1) providing a Lewis acid, such as in particular boron tribromide (BBr₃), aluminum trichloride (AlCl₃), aluminum tribromide (AlBr₃), boron trifluoride diethyl etherate (BF₃*OEt₂) or tin tetrachloride (SnCl₄), or a Brønsted acid, such as in particular hydrogen iodide (HI) or hydrogen bromide (HBr);
c1) reacting the substance according to step al) with the substance from step b1) to afford the substance of formula C1) 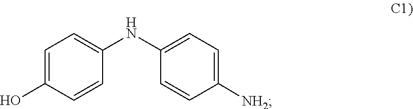 [SMILES: Nc1ccc(Nc2ccc(O)cc2)cc1]
d1) providing methyl isobutyl ketone (MIBK) and hydrogen;
e1) reacting the substance of formula C1) with methyl isobutyl ketone (MIBK) from step d1) to afford the substance of formula I)

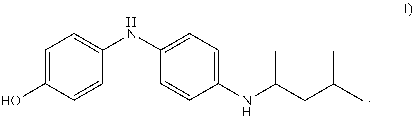 [SMILES: CC(C)CC(C)Nc1ccc(Nc2ccc(O)cc2)cc1]

20. The rubber mixture of claim 13, the rubber mixture is used as an aging stabilizer and/or antiozonant in particular in vehicle tires and/or technical rubber articles, such as in particular an air spring, bellows, conveyor belt, belt, drive belt, hose, rubber band, profile, a seal, a membrane, tactile sensors for medical applications or robotics applications, or a shoe sole or parts thereof and/or oils and/or lubricants.

21. The rubber mixture of claim 13, the rubber mixture is used a rubber article, in particular an air spring, a bellows, conveyor belt, belt, drive belt, hose, rubber band, profile, a seal, a membrane, tactile sensors for medical applications or robotics applications, or a shoe sole or parts thereof.

22. The rubber mixture of claim 13, the rubber mixture is used in oils and lubricants, such as in particular fuels or fluids for engines.

23. The rubber mixture of claim 13, the rubber mixture is incorporated as a dye in fibers and/or polymers and/or paper and/or in paints and coatings.
